(19) United States
(12) Patent Application Publication
(10) Pub. No.: US 2004/0138681 A1
(43) Pub. Date: Jul. 15, 2004

(54) Surgical-clip applicator

(76) Inventor: Arnold Pier, Heinsberg (DE)

Correspondence Address: Diller Ramik & Wight, Merrion Square, Suite 101, 7345 McWhorter Place, Annandale, VA, 22003, US

(21) Appl. No.: 10/475,301

(22) PCT Filed: Apr. 24, 2002
(86) PCT No.: PCT/EP02/04483

(30) Foreign Application Priority Data
Apr. 25, 2001 (DE) 10120367.5

(51) Int. Cl. A61B 17/10

(52) U.S. Cl. 606/143

(57) ABSTRACT

In a surgical clamp applicator (1) comprising a grip (2) having an actuating means (4); a shaft tube (6) fastened to the grip (2), in which shaft tube (6) a push rod (8) adapted to be shifted in longitudinal direction by the actuating means (4) and returned by spring force is arranged; and an exchangeable head portion (17) arranged at the distal end of the shaft tube (6), said head portion (17) compring a clamp magazine (12) receiving a plurality of clamps (10); and a closing element (14) for the clamps (10) arranged at the distal end (16) of the head portion (17), wherein the push rod (8), in the proximal portion (20) of the shaft tube (6) acts upon a spring-loaded piston element (25) arranged in the head portion (17), said piston element (25) being connected with a transport slide (24) for the clamps (10) in the clamp magazine (12), it is provided that between the push rod (8) and the piston element (25) a snap-in element (54) is arranged.

The invention relates to a surgical clamp applicator according to the precharacterizing part of claim 1.

Such clamp applicators are e. g. used for laparoscopic surgery but can also be employed for open surgery.

In the laparoscopic and/or the endoscopic surgery the clamp applicator is inserted through a trocar sleeve e. g. into the abdominal region.

A clamp applicator known from DE-A-195 04 002 comprises a grip having an actuating means; a shaft tube fixed to the grip, in which shaft tube a push rod is arranged which is adapted to be shifted in longitudinal direction by the acutating means and returned by spring force; and an exchangeable head portion arranged at the distal end of the shaft tube, said head portion comprising a clamp magazine receiving a plurality of clamps; and a closing element for the clamps disposed at the distal end of the head portion, wherein the push rod, in the proximal portion of the shaft tube, acts upon a spring-loaded piston element arranged in the head portion, which element is connected with a transport slide for the clamps in the clamp magazine.

The head portion containing a clamp magazine as well as a transport mechanism and a closing element is releasably coupled with the proximal portion of the shaft tube. Thus, after removal of the distal head portion, the clamp applicator comprises only a few mechanical parts which can be easily sterilized. The head portion, from which the clamps have been removed, can be disposed of. After sterilization of the clamp applicator a new sterile head portion is attached such that the sterilizable clamp applicator can be reinserted. The elements not suitable for sterilization are accommodated in the disposable clamp magazine in the head portion. The fact that only the head portion of the shaft tube and not the entire clamp applicator has to be disposed of results in a considerable cost reduction with regard to the use of clamp applicators.

The push rod of the proximal portion of the shaft tube acts upon a spring-loaded piston element, wherein the piston element is connected with a transport slide for the clamps in the clamp magazine. The push rod actuates the piston element in the head portion, which piston element is spring-loaded and automatically moves back due the spring load acting upon it when the push rod moves in proximal direction. Thus it is only required that the push rod exerts a force in distal direction upon the piston element.

The clamp magazine is adapted to accommodate clamps which can be absorbed by the human body.

The disadvantage of the known clamp applicators is that maloperation cannot be prevented when the grip of the clamp applicator is released before the farthermost clamp has been closed. In this case another clamp is pushed forward when the clamp applicator is actuated again although the farhermost clamp is not yet closed and not yet applied.

It is an object of the invention to provide a surgical clamp applicator which prevents maloperation.

This object is achieved with the features of claim 1.

According to the invention, a snap-in element is preferably provided between the push rod and the piston element. The snap-in element allows the operator to reversibly describe a certain feed path wherein no maloperation can occur. In the first feed area the actuating means of the clamp applicator can be released without another clamp being moved forward when the clamp applicator is actuated again. If the push rod is moved in distal direction over the first feed area, the snap-in piece can snap into a given feed position and show the operator that a “point of no return” has been reached where the clamp must be completely closed. When the snap-in piece snaps in the operator can feel this snapping motion on the grip such that maloperation of the instrument can be prevented to a large extent. Upon further movement of the push rod in distal direction the clamp is closed and the snap-in element returns to its initial position. The snap-in piece thus reliably prevents maloperation of the instrument and further allows the clamp applicator to be reversibly actuated in a first feed area, wherein the farthermost clamp can be opened again upon proximal movement of the push rod. Even when the farthermost clamp is already disengaged from the following clamp, proper actuation is ensured.

In the clamp magazine a plurality of clamps arranged in one after the other are guided in a clamp shaft and engage each other. The clamp magazine can receive a number of clamps sufficient for an operation. If the number of clamps does no suffice for an operation, it is possible to replace an empty head portion by a new one during the operation. Due to the fact that the clamps in the clamp shaft engage each other all clamps in the clamp magazine can be jointly transported.

A locking tongue retains the clamps in the clamp magazine in position during movement of the transport slide in proximal direction. The locking tongue thus prevents the clamps in the clamp magazine from being moved in proximal direction during the return movement of the piston element and the transport slide connected therewith.

It is provided that the locking tongue is spring-deflected during transport of a clamp in distal direction. The locking tongue can thus allow a clamp transported in distal direction to pass through and merely prevents the movement of the clamp in proximal direction.

The transport slide comprises a slide head whose front face presses against a proximal end of a clamp.

During movement in proximal direction the transport slide can be displaced into a groove of the head part when it runs over a clamp in the clamp magazine. In this manner the transport slide can grip behind the following clamp in the clamp magazine after ejection of the farthermost clamp while the locking tongue retains said following clamp in position.

The clamps comprise projections arranged on the outside of the legs, said projections engaging recesses matching the projections and provided on the inside of the legs of another clamp arranged proximally. Upon actuation of the actuating means the transport slide pushes the farthermost clamp in distal direction. All following clamps are coupled with each other via the projections and recesses and are fed forward by one position when the transport slide is moved in distal direction.

A second clamp located in proximal direction in the clamp magazine is held by the locking tongue, whereby all following clamps are retained in their position.

The snap-in element can snap in at a location distal to the first feed area in which a reversible movement is possible at a feed position of the transport slide at which the locking tongue grips behind the proximal end of the second clamp. Snapping-in of the snap-in element thus takes place at the “point of no return” at which the locking tongue fixes the seond clamp in its position. A return stroke of the snap-in element and the transport slide is thus prevented such that the transport slide cannot move the second clamp in distal direction when the actuating means at the grip is actuated again. Thus the transport slide is prevented from being moved in proximal direction to such an extent that that it can grip the second clamp when it is moved again in distal direction.

The head portion comprises an upper shell and a lower shell which are preferably made by injection molding.

The head portion can be connected with the proximal end of the shaft tube via a bayonet catch.

The clamp shaft is, in the distal area of the head portion, inclined by approximately 7° to 15°, preferably 9° relative to the longitudinal axis of the shaft tube. The inclination of the clamp shaft facilitates the handling of the clamp applicator during an operation.

The closing element consists of a narrowed section at the distal end of the clamp shaft, in which narrowed section the legs of the farthermost clamp are closed when they are fed forward. Such a closing element does not require any movable parts besides the transport slide and is particularly suitable for clamps made of plastic material.

Instead of the last clamp a locking part is arranged in the clamp magazine. The locking part can accommodate the last clamp and prevents maloperation of the clamp applicator. The operator is thus informed that there are no clamps in the clamp magazine.

The proximally projecting web portion of the locking part further prevents the transport slide from gripping the locking part.

The shaft tube is preferably rotatably supported on the grip, which allows the shaft tube including the head portion to be rotated by 360° during the application process.

At least in the area of the clamp magazine the head portion may comprise an inspection window through which it can be seen during an operation whether there are enough clamps in the clamp magazine. The inspection window can further be provided with a numbering such that the operator can directly read the number of clamps available.

The actuating means comprises a release lever supported on the grip, said release lever acting upon the push rod. The release lever is a component of the push rod drive by means of which the piston element can be moved in distal direction.

The force of a leaf spring can be applied against the force of the return movement of the push rod to the release lever, said leaf spring defining a shiftable stop for the release lever. Upon movement of the release lever counter to the normal direction of actuation the stop defined by the leaf spring can be pushed back until the push rod is released. The push rod can then be taken out of the shaft tube and the actuating means, which allows the clamp applicator to be easily dismantled for sterilization purposes.

Hereunder embodiments of the invention are explained in detail with reference to the drawings in which:

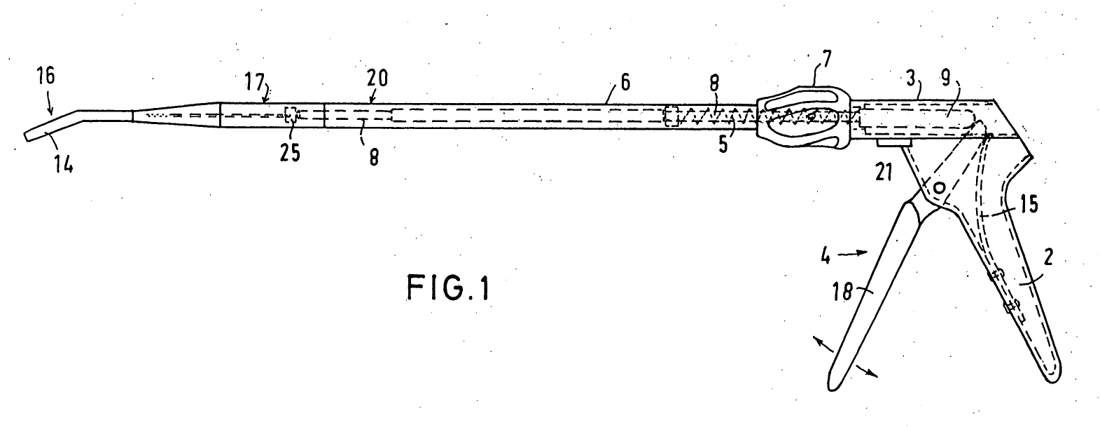

FIG. 1 shows a side view of a clamp applicator.

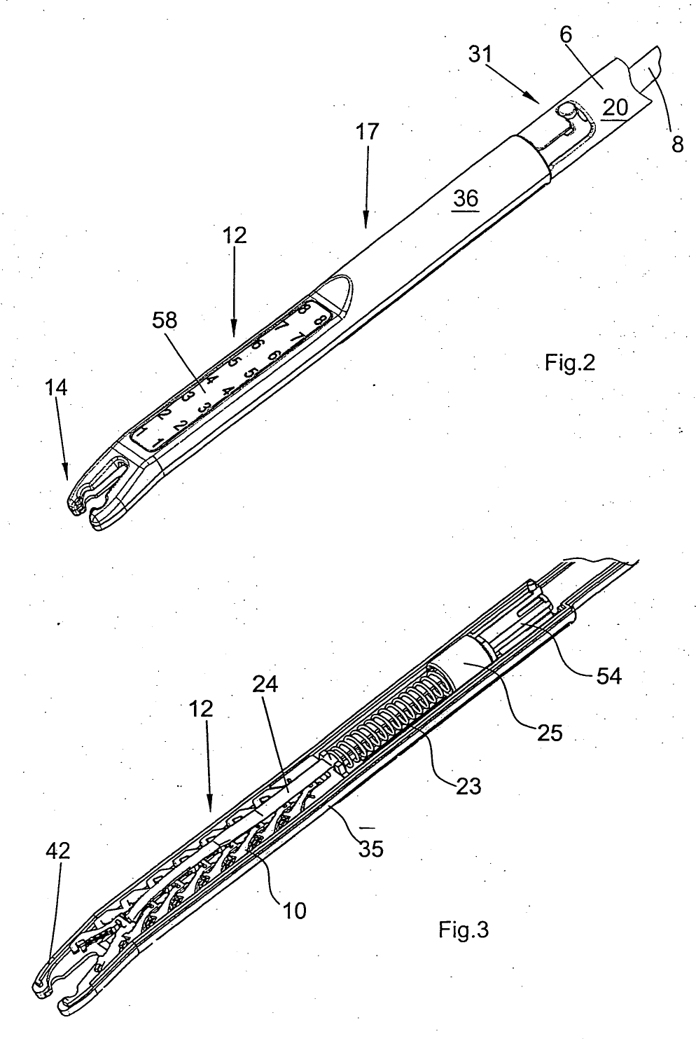

FIG. 2 shows a perspective view of a head portion.

FIG. 3 shows the head portion without its upper shell.

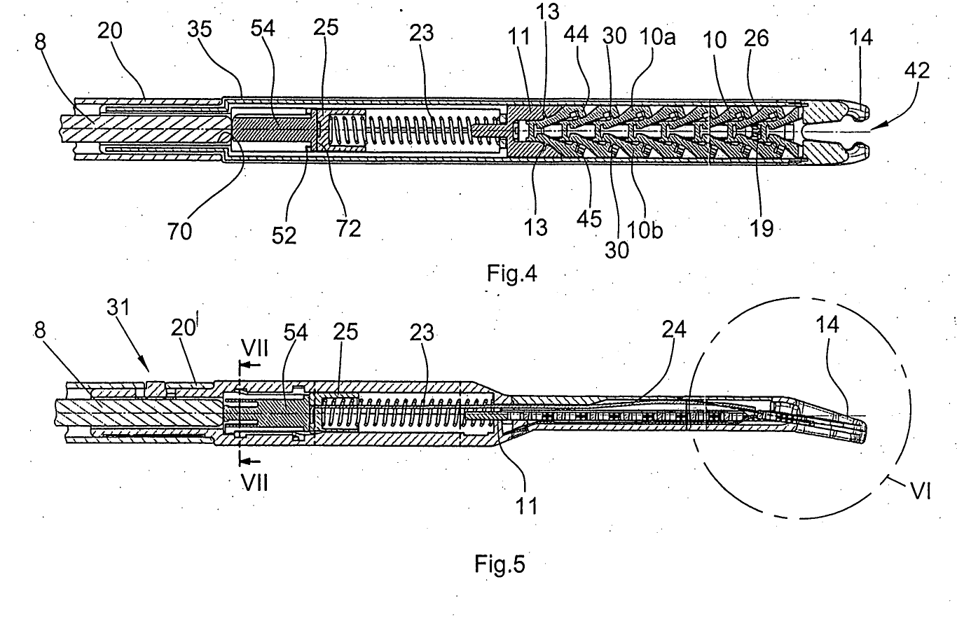

FIG. 4 shows a cross-section of the head portion.

FIG. 5 shows a cross-section of the head portion in a plane offset by 90° relative to the view shown in FIG. 4.

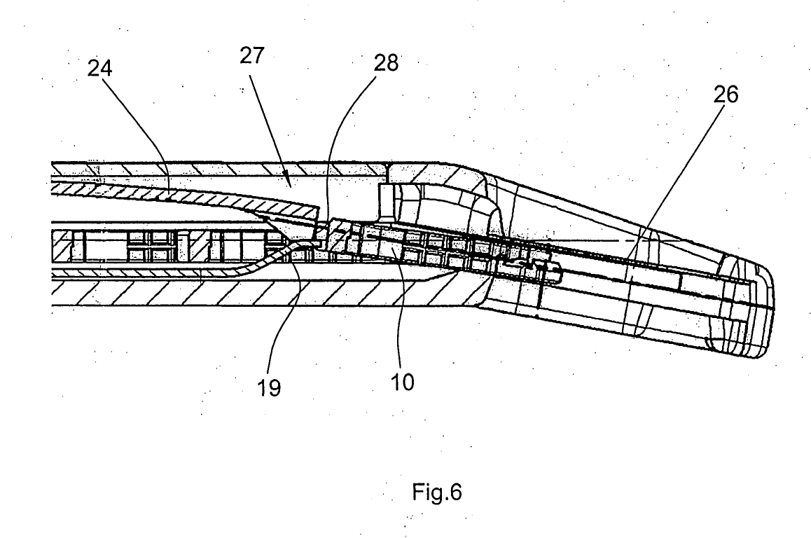

FIG. 6 shows an enlarged detail of FIG. 5.

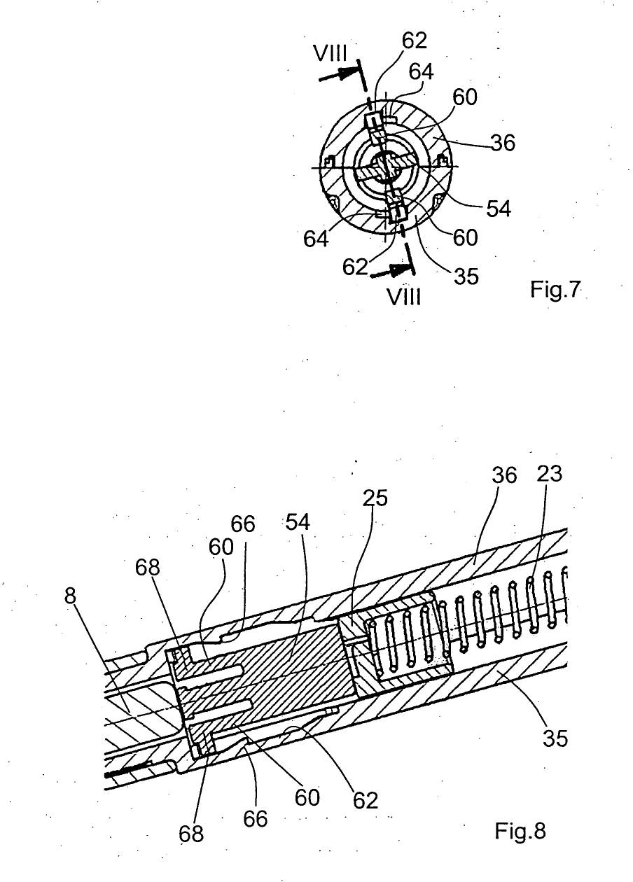

FIG. 7 shows a cross-section along line VII-VII of FIG. 5.

FIG. 8 shows a longitudinal section along line VIII-VIII of FIG. 7.

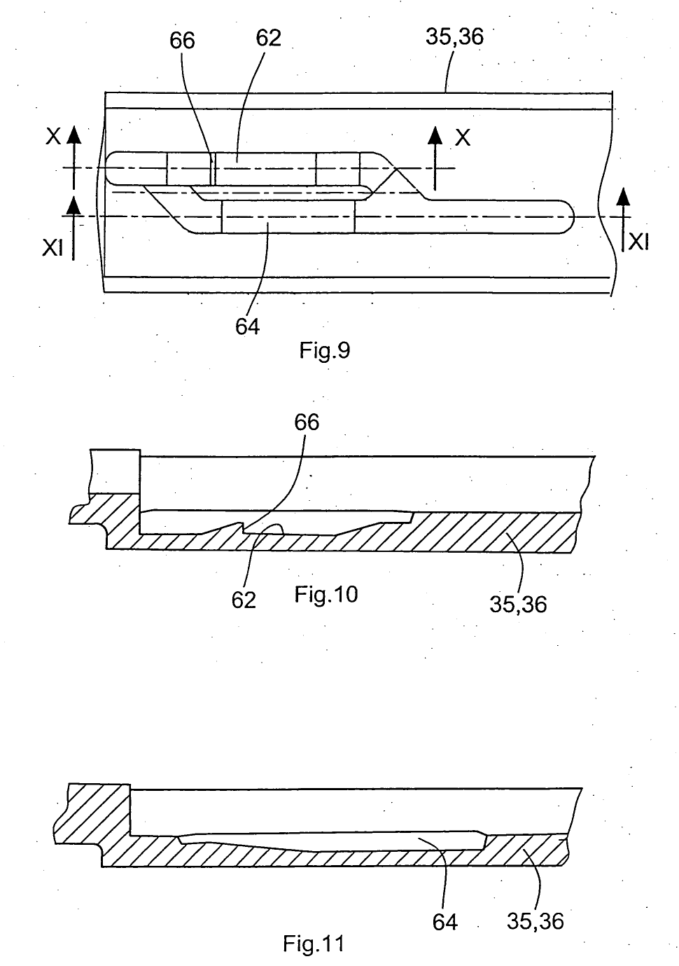

FIG. 9 shows a groove profile in the upper and the lower shell.

FIG. 10 shows a section along line X-X of FIG. 9.

FIG. 11 shows a section along line XI-XI of FIG. 9.

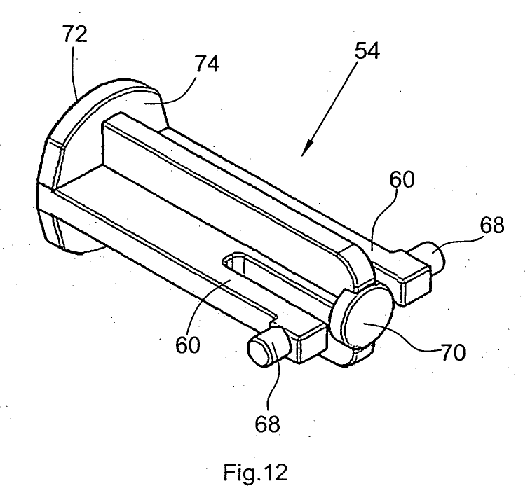

FIG. 12 shows a snap-in element.

The clamp applicator 1 shown in FIG. 1 comprises a grip 2 in which an actuating means 4 essentially having a release lever 18 and a push rod 8 is supported. By means of the release lever 18 the push rod 8 can be pushed towards the distal end 16 of the clamp applicator 1. Upon termination of the actuating process the push rod 8 is pushed back into its initial position with the aid of a return spring 5. The ram 9 of the push rod 8 bears upon the lever arm 21 of the release lever 18, wherein a leaf spring 15 applies a counter-torque to the lever arm 21. The leaf spring 15 defines a stop. The leaf spring 15 can be rotated counter to the normal direction of actuation, whereby the lever arm 21 is moved further to the right in the drawing, which allows the push rod 8 to be removed from the shaft tube 6 for sterilization purposes.

The shaft tube 6 is rotatably supported in the housing 3 of the grip 2 and can be rotated with the aid of a grip ring 7. Together with the shaft tube the push rod 8 can be rotated, which push rod 8 is of rotationally symmetrical configuration at its proximal and its distal end. The shaft tube accommodates at its distal end a head portion 17 with a clamp magazine 12 which comprises a clamp shaft 26 and a transport slide 24. The shaft tube 6 accommodates the head portion 17 as an exchangeable unit which is releasably connected with a proximal portion 20 of the shaft tube 6. The releasable connection is preferably a bayonet catch.

The shaft tube 6 is attached to rotate with the grip ring 7 and coupled to the housing 3 via the grip ring 7.

The push rod 8 has a circular cross-section and extends in proximal direction through the grip ring up to and into the housing 3, wherein the push rod 8 comprises a ram 9 at its proximal end which pushes against the lever 21 located in the housing 3. The return spring 5 consists of a helical spring which surrounds the proximal end of the push rod 8 and whose one end bears against the front end of the ram 9 opposite the lever arm 21 and whose other end bears against a disk ring 29 which is shiftable on the push rod 8 and fastened to the shaft tube 6. The ram 9 is e. g. screwed onto the proximal end of the push rod 8.

The clamp applicator 1 can be completely dismantled with a few movements. As has been previously said, the head portion 17 plus the closing element 14 and the clamp magazine 12 can be removed. When fastening means have been loosened, the shaft tube 6 can be separated from the housing 3.

The head section is preferably made from plastic material and is a disposable article which cannot be resterilized. The remaining elements of the clamp applicator 1 can be dismantled and have a simple structure such that resterilization is possible without any problems. Thus, apart from the head section 17, the clamp applicator 1 is preferably made from metal.

The head section 17 is perferably produced as an injection molded part made of plastic material and comprises an upper shell 36 and a lower shell 35 which can be ultrasonically welded to each other.

In the upper shell 36 of the head portion 17 an inspection window 58 can be provided in the area of the clamp magazine 12, through which the number of clamps 10 in the clamp magazine 12 can be checked.

A numbering on the inspection window 58 allows the number of clamps 10 available to be read.

The shaft tube has a circular cross-section with a maximum diameter of approximately 10 to 15 mm, preferably 10 mm. It is an essential advantage of this configuration that such a small diameter of the cross-section can be achieved.

Instead of the last clamp 10 a locking part 11 can be provided in the clamp magazine 12, which locking part 11 can accommodate the last clamp 10. Said locking part 11 is adapted to prevent the clamp applicator 1 from being actuated when no clamps 10 are available. Since the locking element 11 cannot leave the closing element 14, the operator knows when the clamp magazine 12 is empty.

In the head portion 17 a stop 52 can be provided for a piston element 25 which is spring-loaded in proximal direction. Said piston element 25 is pressed by a compression spring 23 against a snap-in element 54 which may distally bear against the stop 52 and which proximally bears against the push rod 8. The distal end of the compression spring 23 is supported on the clamp magazine 12. To the piston element 25 a transport slide 24 is fastened which is adapted to transport the clamps 10 contained in the clamp magazine 12.

Upon movement of the push rod 8 in distal direction the snap-in element 54 and the piston element 25 are in the same manner moved in distal direction by the transport slide. The piston element 25 is unrotatably guided.

The head portion 17 with the clamp magazine 12 may comprise two halves, namely a lower shell 35 and an upper shell 36. Between the upper shell 36 and the lower shell 35 of the clamp magazine 12 a clamp shaft 26 is kept free in which a plurality of clamps 10 are arranged one behind the other and in engagement with each other.

As can be seen from FIG. 6 the transport slide 24 comprising a slide head 27 bears at its distal end against the proximal end of the farthermost clamp 10 in the clamp magazine 12. As shown in FIG. 6, a locking tongue 19 fastened to the lower shell 35 of the clamp magazine extends upwards into the clamp shaft 26. The distal end of the locking tongue 19 bears against the proximal end of the second clamp in the clamp magazine 12 and prevents all clamps in the clamp magazine from sliding back during the return movement of the transport slide in proximal direction. If the transport slide 24 is moved in distal direction upon actuation of the push rod 8, the legs 10a and 10b of the clamp 10 are closed by the closing element 14 which defines a narrowed section 42 at the distal end of the clamp magazine 12. Due to the forward movement of the farthermost clamp 10 the clamp is automatically closed, wherein projections 44 extending from the legs 10a,10b are interlocked with complementary projections 45 of the opposite legs 10a,10b such that the clamp is closed and simultaneously ejected from the clamp shaft 26. During this process the projections on the outside of the legs 10a,10b are disengaged from the recesses 30 of the clamp 10 which follows in the proximal direction. After ejection of the farthermost clamp 10 the transport slide 24 can move backwards during movement of the push rod 8 in proximal direction due to the effect of the compression spring 23. The slide head 27 can be moved upwards due to an inclination on its proximal side and a groove 22 in the upper part 36 of the clamp magazine 12 when it slides over that clamp which is now the farthermost clamp in the clamp magazine. 12. The locking tongue 19 retains all clamps 10 in the clamp magazine 12 in their position. During ejection of the front clamp 10 the following clamp has moved forward in distal direction to such an extent that the locking tongue 19 snaps in behind the clamp which is the second clamp in proximal direction of the clamp magazine 12.

The last clamp 10 in the clamp magazine 12 is in engagement with a locking part 11 which serves for preventing another releasing process to show the operator that the clamp magazine 12 is empty and must be replaced by a new one.

The function of the snap-in element 54 is shown in a perspective view in FIG. 12 and is described hereunder:

The proximal front face 70 of the snap-in element 54 bears against the distal end of the push rod 8. At the distal end of the snap-in element 54 a flange 74 is provided which bears against the piston element 25 and grips behind a stop 52, as shown in FIG. 4. The snap-in element 54 comprises two spring portions 60 located diametrically opposite each other and having radially projecting noses 68 at their proximal ends which can engage the guide grooves 62,64 defined in the two shell parts 35,36. The guide grooves 62,64 are shown in detail in FIGS. 9 to 11.

FIG. 8 shows the noses 68 engaging the groove 62 in the initial position of the snap-in element 54. The grooves 62 located diametrically opposite each other in the two shell parts comprise a snap-in projection 66 behind which the radially projecting noses 68 of the spring portions 60 can snap in. Up to snapping-in of the noses 68 distally behind the snap-in projections 66 the movement of the push rod 8, the snap-in element 54, the piston element 25, the transport slide 24 connected with the latter and the distally farthermost clamp 10 is reversible, i. e. upon return movement of the release lever 18 in proximal direction all elements return into their initial position. When the release lever is actuated again, the transport slide 24 cannot grip the clamp located proximally behind the farthermost clamp 10 such that maloperation, where two clamps are simultaneously moved in distal direction, is prevented. If the feed movement has been performed to such an extent that the snap-in element 54 snaps in, wherein the noses 68 arrive at a location distally in front of the snap-in projection 66, the snap-in element 54 cannot return into its initial position when the release lever 18 is released, which prevents the transport slide 24 with its slide head 27 from being moved behind the second clamp 10 when the farthermost clamp 10 has been moved forward too far in distal direction. When the release lever 18 is actuated again, only the farthermost clamp 10 is moved in distal direction. In this connection, it is irrelevant whether the farthermost clamp 10 has already been brought in engagement with the second clamp during a previous actuation process. The snap-in element 54 can return into its initial position only when the noses 68 have changed over from the guide groove 62 to the guide groove 64, as shown in FIG. 9, and have been moved forward to such an extent in distal direction that the farthermost clamp element 10 has been closed and ejected. In this manner, maloperation is reliably prevented.

FIG. 11 shows a cross-section along the guide groove 64 essentially extending in parallel to the guide groove 62, said guide groove 64 returing the snap-in element 54 into its initial position.

Of course, other embodiments of a snap-in element 54 can be used which ensure that the snap-in element can move from an initial position up to a snap-in position back to the initial position via the feed path and where the snap-in element being located distally from the snap-in position can only be returned into the initial position if and when the distally farthermost clamp is closed.

1. Surgical clamp applicator (1) comprising
a grip (2) having an actuating means (4),
a shaft tube (6) fastened to the grip (2), in which shaft tube (6) a push rod (8) adapted to be shifted in longitudinal direction with the aid of the actuating means (4) and returned by spring force is arranged, and
an exchangeable head portion (17) arranged at the distal end of the shaft tube (6), said head portion (17) comprising a clamp magazine (12) receiving a plurality of clamps (10), and a closing element (14) for the clamps (10) arranged at the distal end (16) of the head portion (17), wherein the push rod (8), in the proximal portion (20) of the shaft tube (6), acts upon a spring-loaded piston element (25) arranged in the head portion (17), said piston element (25) being connected with a transport slide (24) for the clamps (10) in the clamp magazine (12),
characterized in that
between the push rod (8) and the piston element (25) a snap-in element (54) is arranged.

2. Clamp applicator according to claim 1, characterized in that the snap-in element (54) allows from an initial position up to a snap-in position via the feed path a return to the initial position, and the snap-in element (54), after having reached the snap-in position, can be returned into the initial position only when the distally farthermost clamp (10) has been closed.

3. Clamp applicator according to claim 1 or 2, characterized in that in the clamp magazine (12) a plurality of clamps (10) arranged one after the other are guided in a clamp shaft (26) and engage each other.

4. Clamp applicator according to one of claims 1 to 3, characterized in that a locking tongue (19) retains the clamps (10) in their position during movement of the transport slide (24) in proximal direction when the farthermost distal clamp (10) has been closed and ejected.

5. Clamp applicator according to claim 4, characterized in that the snap-in element (54) snaps in at a location distal to the first feed area at a feed position of the push rod (6) at which the locking tongue (19) grips behind the proximal end of the second clamp (10).

6. Clamp applicator according to one of claims 1 to 3, characterized in that the transport slide (24) comprises a slide head (27) whose distal front face (28) presses against a proximal end of a clamp (10).

7. Clamp applicator according to one of claims 1 to 6, characterized in that the transport slide (24) during movement in proximal direction can be displaced when it runs over a clamp (10).

8. Clamp applicator according to one of claims 1 to 7, characterized in that the clamps (10) comprise projections (13) arranged on the outside of the legs (10a,10b), said projections (13) engaging recesses (30) matching the projections and provided on the inside of the legs (10A,10B) of another clamp (10) arranged in proximal direction.

9. Clamp applicator according to one of claims 1 to 8, characterized in that the transport slide (24) acts upon the farthermost distal clamp (10) in the clamp magazine (12).

10. Clamp applicator according to one of claims 4 to 9, characterized in that the proximal end of the clamp (10) which is the second one in proximal direction in the clamp magazine (12) bears against the locking tongue (19).

11. Clamp applicator according to one of claims 1 to 10, characterized in that the head portion (17) comprises an upper shell (36) and a lower shell (35).

12. Clamp applicator according to one of claims 1 to 10, characterized in that the head portion (17) is connected via a bayonet catch with the proximal portion (20) of the shaft tube (6).

13. Clamp applicator according to one of claims 2 to 12, characterized in that the clamp shaft (26), in the distal portion of the head portion (17), is inclined by approximately 7° to 15°, preferably 9° relative to the longitudinal axis of the shaft tube (6).

14. Clamp applicator according to one of claims 2 to 13, characterized in that the closing element (14) consists of a narrowed section (42) at the distal end of the clamp shaft (26), in which narrowed section (42) the legs (10a, 10b) of the farthermost clamp (10) are closed during the feed movement.

15. Clamp applicator according to one of claims 1 to 14, characterized in that instead of the last clamp (10) in the clamp magazine (12) a locking part (11) comprising a proximally projecting web portion (56) is arranged.

16. Clamp applicator according to one of claims 1 to 15, characterized in that the head portion (17) comprises an inspection window (58) at least in the area of the distal end of the clamp magazine (12).

17. Clamp applicator according to one of claims 1 to 16, characterized in that the shaft tube (6) is rotatably supported in the grip (2).

18. Clamp applicator according to one of claims 1 to 17, characterized in that the actuating means (4) consists of a release lever (18) supported on the grip (2), which release lever (18) acts upon the push rod (8).

19. Clamp applicator according to claim 18, characterized in that a leaf spring (15) act against the force of the return movement of the push rod (8) upon the release lever (18), said leaf spring (15) defining a shiftable stop for the release lever (18).

20. Clamp applicator according to one of claims 2 to 19, characterized in that at least one spring portion (60) of the snap-in element (54) engages at least one guide groove (62,64), and the guide groove (62) comprises at least one recess or one snap-in projection (66) which can be engaged by a nose (68) of the spring portion (60) in the snap-in position of the snap-in element (54).
